(19) United States
(12) Patent Application Publication
(10) Pub. No.: US 2012/0051969 A1
(43) Pub. Date: Mar. 1, 2012

(54) STETHOSCOPE DISINFECTION DEVICE

(75) Inventors: Dean Nahman, Jerusalem (IL); Amos Cahan, Mevaseret Zion (IL)

(73) Assignee: HADASIT MEDICAL RESEARCH SERVICES AND DEVELOPMENT

(21) Appl. No.: 13/320,109

(22) PCT Filed: May 13, 2010
(86) PCT No.: PCT/IL10/00385

§ 371 Date: Nov. 11, 2011

Related U.S. Application Data
(60) Provisional application No. 61/177,662, filed on May 13, 2009.

(51) Int. Cl. A61L 2/18; A61L 2/23

(52) U.S. Cl. 422/28; 422/300

(57) ABSTRACT

The present invention provides a system adapted to disinfect a stethoscope head wherein said system comprises: (a) a plurality of disposable pads having adhesive and disinfection agents absorbed therein, said pads being adapted to adhere to and disinfect said stethoscope head; and, (b) a portable dispenser adapted for serially dispensing said disposable pads, said dispenser comprising: a. a front face having an aperture adapted to accommodate said stethoscope head, and a back face; b. a cover for encasing said aperture, said cover being at least partially removable from said front face during use of said system; c. a container adapted to accommodate said disposable pads.

### FIELD OF THE INVENTION

The present invention relates to a device for disinfection of stethoscopes.

### BACKGROUND OF THE INVENTION

The stethoscope is a device for amplifying and transmitting various bodily sounds such as those of the heart, lungs, intestines, and blood flow in arteries and veins. In the prior art shown in FIG. 1 we see a stethoscope of a common variety with earpieces 101 for listening to bodily sounds, eartubes 102 to transmit the sound from flexible tubing 103, and a chestpiece or ‘head’ 104.

The cleanliness of the stethoscope head is of great concern, especially in hospitals, where infectious agents can pass easily from one patient to the next through contact with contaminants on a doctor's stethoscope. The stethoscope head is particularly prone to contamination since it comes into physical contact with each patient. To date, no device has become universally accepted for performing stethoscope disinfection, so as to prevent unnecessary spreading of infections. To be practical, such a device has to be very easy and convenient to use, not requiring the doctor any significant amount of time or effort.

U.S. Pat. No. 5,365,023 to Lawton relates to an elastic, disposable cover for the head of a stethoscope. The disk-shaped cover is manufactured from latex or other material that is sufficiently elastomeric to allow the cover to be stretched over the stethoscope head during installation, and snap back to remain taut after installation and during use of the stethoscope. The outer edge of the disk is preferably rolled, and the disk is preferably pre-shaped in a shallow parabola, to further ease installation and removal. Since the cover may be fit to a wide variety of stethoscope head types, it is more practical and easier to use than prior stethoscope covers. However we note that the provision is for a cover for the stethoscope head, as opposed to a method for sterilization thereof. The cover provided would have to itself either be sterilized or replaced after each use to provide a sterile contact surface for the stethoscope head. Similarly U.S. Pat. No. 6,467,568 to Kemper relates to a stethoscope cover and dispenser therefore. The stethoscope cover comprises any material which acts as an effective, inexpensive and acoustically transmissive barrier means for easy application and removal from the head of a stethoscope. The barrier means comprising the stethoscope cover comprises folds, gathers or crimping at the marginal edges or medial portions thereof, providing flexibility, extensibility or elasticity to the cover, thus facilitating easy application and removal of the stethoscope cover from the head of the stethoscope. The folds, crimping or gathers on the marginal edges of the covers also assist in the retention of the stethoscope cover on the stethoscope head during use. As in the previous case however, provision is made only for a cover for the stethoscope head, as opposed to a method for sterilization thereof. The cover provided would have to itself either be sterilized or replaced after each use to provide a sterile contact surface for the stethoscope head.

Similarly U.S. Pat. No. 6,206,134 to Stark et al relates to a cover for a stethoscope head having a discontinuous layer of peelable adhesive in a predetermined pattern for detachable affixing to the head of the stethoscope including the diaphragm. The pattern has a plurality of spaced adhesive segments and provides air flow passages from regions between the cover and the diaphragm to regions external the stethoscope to prevent the formation of air pockets and bubbles therebetween. U.S. Pat. No. 6,499,560 to Lang et al discloses a disposable diaphragm assembly for use with all types of conventional stethoscopes and a universal retainer ring for retro-fitting conventional stethoscopes to accept the disposable diaphragm assembly. The invention further relates to a stationary dispenser for the disposable diaphragm assemblies from which the assemblies may be loaded onto either retrofitted conventional stethoscopes or the novel stethoscope of this invention without the assembly being contaminated by the hands of medical personnel. In both of these cases however, provision is again made only for a cover for the stethoscope head, as opposed to a method for sterilization thereof. To allow sterile use of the stethoscope, the cover provided would have to itself either be sterilized or replaced after each use to provide a sterile contact surface for the stethoscope head.

In similar fashion U.S. Pat. No. 7,117,971 to Cornacchia relates to a stethoscope cover applicator for facilitating application of a protective membrane to the stethoscope that includes a membrane member configured to be selectively secured to a diaphragm of the stethoscope to inhibit contact between the skin of the patient and the stethoscope. An axle is couplable to the membrane member and the membrane member is rolled around the axle. A base member configured to engage a support surface receives the axle to facilitate unrolling of the membrane member from the axle. Again however, provision is made only for a cover for the stethoscope head, as opposed to a method for sterilization thereof. The cover provided would have to itself either be sterilized or replaced after each use to provide a sterile contact surface for the stethoscope head.

U.S. Patent Application No. 2006/0147339 to Hunter; Charles Eric et al. relates to a method and apparatus for ultraviolet sterilization. The apparatus includes a housing having an opening through which objects can be inserted for sterilization, a killing chamber having an inner surface that is substantially reflective at ultraviolet wavelengths; and at least one ultraviolet radiation source inside the housing. The method and apparatus are suited for use with medical tools, dental tools, body art tools, and baby feeding related objects.

Similarly U.S. Patent Application No. 2002/0146343 to Jenkins, Geoffrey H. et al. discloses an ultraviolet sterilization system, as does U.S. Patent Application No. 2002/0162972 to Pleet and No. 2005/0254992. Such UV-based sterilization devices are generally designed to be of a “table-top” type construction, and are not adapted to be worn by the user since they require bulky power supplies and furthermore emit radiation harmful to living organisms. The devices are thus more complicated and expensive than what is required for sterilization of a relatively small stethoscope head, and in particular cannot be worn by the user in a convenient fashion.

U.S. Pat. No. 7,282,186 to Lake, Jr., et al. relates to a decontamination device for decontaminating medical apparatus including a housing and a dispenser within the housing for contacting a portion of the medical apparatus with a decontaminating compound when the portion of the medical apparatus is placed within the housing. A structure for removably engaging the housing to the medical apparatus is provided. The decontamination device can be used with many different medical apparatus, such as stethoscopes. A method for decontaminating medical apparatus is also provided. This method while in principle providing a portable means for disinfection, suffers from a number of drawbacks. The housing must be manually removed before each examination and re-attached after each exam. The dispenser containing disinfectant solution will either have to be disposed of after each said exam, or reused. If reused, it will eventually dry out as there is no provision for re-filling the dispenser with disinfectant solution.

U.S. Pat. No. 6,018,835 to Schonfeld relates to a cleaning apparatus for a stethoscope. The invention includes a housing defining an interior cleaning chamber and having an opening communicating between an exterior of the housing and the cleaning chamber. The opening in the housing permits the head portion of the stethoscope to be inserted into the cleaning chamber. An apparatus is arranged in the cleaning chamber for disinfecting the head portion of the stethoscope inserted through the opening. However the method, like those using ultraviolet sterilization, is not of a portable nature. Thus a physician making rounds of a plurality of patients would not be able to use the system in a convenient fashion.

Similarly U.S. Pat. No. 5,641,464 provides a cleaning apparatus but one that is not portable to the degree that it is a conveniently wearable device. Each of the aforementioned patents employ different constructions and cleaning mechanisms for achieving stethoscope disinfection. In contrast to these devices, the proposed device has the distinct and valuable advantage that it is designed to be worn by the doctor. Each of the aforementioned patents disclose only the option of a standing device, and make no mention of the possibility of the device being worn by the user, which constitutes a disadvantage for these devices when compared to a device which may be worn by a health-care professional as he or she travels from patient to patient.

Another type of sterilization device, which is designed to be slidably attached to the tube of the stethoscope or held in the pocket of the doctor, is disclosed in U.S. Patent Application No. 2007/0080017. The cited invention utilizes at least one layer of absorbent material that is adapted for holding a sanitizing agent and which contacts the stethoscope head when the device is slid down over the diaphragm. As described in the detailed description of the drawings section of that invention, one preferred embodiment of the invention is a flexible cone shaped pocket that uses foam pads to store a sanitizing agent, such as alcohol gel, in its interior. When the stethoscope head is slid into the device, the foam pads and sanitizing agent inside the device initiate and maintain contact with the stethoscope's diaphragm and bell, thereby sanitizing the diaphragm and bell, and minimizing any cross contamination. The germ killing ability of the device can be increased via massaging of the device while the head is inside the device. The sanitizing pad is preferably attached to the back panel of the device with Velcro™, or other attachment methods such as adhesives, straps or rigid clips. When the stethoscope is not being used the device covers the diaphragm and bell portions of the stethoscope with sanitizing pads that kill germs and prevent the spread of disease between patients that are examined with the stethoscope. When the health care provider needs to use the stethoscope, the device can be slid up a length of the stethoscope to a desired location so that the diaphragm is exposed for use. The device is advantageously produced with a narrow top that frictionally engages with the tube of the stethoscope so that the sanitizing device stays at the desired location until the user pulls the device back down over the diaphragm. In an alternative embodiment, the sanitizing device is designed to be held within the front pocket of a health care provider's shirt or lab coat so that sterilization of the diaphragm and bell are achieved by placing the head portion of the stethoscope in the front pocket. Using the invention, the doctor has to massage the device in order to maximize the germ killing ability of the pad, and the use of multiple pads can become problematic, since, for example, doctor may have to regularly check how much disinfecting agent remains in the pad(s) in order to know when the pad needs to be replaced or massaged. Additionally, the design of the proposed invention requires the practitioner to hold the device in his pocket or affix to the stethoscope tube which may be less convenient than if he was able to wear the device directly on a shirt or jacket.

Hence, a system disinfecting a stethoscope head using an optimum amount of disinfecting agent is with each disinfection and which the healthcare practitioner can wear the device directly on the shirt or jacket is still a long felt need.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which

FIG. 1 presents a stethoscope;

FIG. 2 presents an exploded view of the present invention, a stethoscope disinfection device;

FIG. 3 presents the sequence of steps involved in using the method of the invention;

FIG. 4 present an alternative embodiment of the device using a pump;

FIG. 5 presents an exploded view of the alternative embodiment using a pump;

FIG. 6 presents the sequence of steps involved in using the alternative embodiment using a pump;

FIG. 7 presents an alternative embodiment of the invention using a swiveling cap; and

FIG. 8 presents several alternative embodiments of removable caps.

FIG. 9 presents one embodiment of the invention using pads.

FIG. 10 presents an embodiment of the device using a roll.

FIG. 11 presents another view of the device of FIG. 10.

FIG. 12A-D present a spring-loaded embodiment of the device.

FIG. 13A-C present a clip-on embodiment of the device.

FIG. 14A, B present an embodiment of the device using a sliding lid.

FIG. 15A-C present another embodiment of the device using a sliding lid and perforated dispenser.

FIG. 16A-C present embodiments of the device using sliding perforated members.

### SUMMARY OF THE INVENTION

It is one object of the present invention to provide means adapted to disinfect a stethoscope head (202) wherein said system comprises: (a) a plurality of disposable pads (204) having adhesive and disinfection agents absorbed therein, said pads being adapted to adhere to and disinfect said stethoscope head; and (b) a portable dispenser adapted for serially dispensing said disposable pads, said dispenser comprising:
a. a front face (203) having an aperture adapted to accommodate said stethoscope head (201), and a back face (207);
b. a cover (202) for encasing said aperture, said cover being at least partially removable from said front face during use of said system;
c. a container (205) adapted to accommodate said disposable pads (204).

It is another object of the present invention to provide the system as defined above, additionally comprising a spring (206) coupled to said back face and to said container, adapted to push said disposable pads in the direction of said stethoscope head.

It is another object of the present invention to provide the system as defined above, additionally comprising a clip (208) coupled to said back face, adapted to attach the device to a physician pocket.

It is another object of the present invention to provide the system as defined above, additionally comprising a clip coupled to said back face, adapted to attach the device to a stethoscope head.

It is another object of the present invention to provide the system as defined above, wherein said disposable pads are impregnated with a disinfecting agent taken from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

It is another object of the present invention to provide the system as defined above, wherein said pads are impregnated with an adhesive agent selected from a group including elastomers, polymers, thermoplastics, thermosetting adhesives, cement, mucilage, glue, paste, cyanoacrylates, methacrylates, urethanes, epoxies, serum albumen, agar, algin, collagen, casein, starch, resins, and bioadhesives, and combinations thereof.

It is another object of the present invention to provide a system adapted to disinfect a stethoscope head with disinfecting agents; wherein said system comprising:
a. an entrance portion (509) adapted to accommodate said stethoscope head;
b. an exit portion (510) adapted to extract said stethoscope head; said entrance portion and exit portion are in physical communication so as to enable said stethoscope head to be conducted from said entrance portion to said exit portion;
c. a reservoir (504) accommodating said disinfecting agents;
d. a spraying mechanism adapted to spray said stethoscope head with said disinfecting agent when said stethoscope head is conducted from said entrance portion to said exit portion.

It is another object of the present invention to provide the system as defined above, wherein said spraying mechanism comprising:
a. a spray pump (505) having an inlet pipe coupled to said reservoir (504); and, an outlet pipe having at least one nozzle (509);
b. a spring loading mechanism in communication with said spray pump adapted to compress said spray pump when said stethoscope head is conducted from said entrance portion to said exit portion;
c. a trigger lever (506) in liquid communication with said spring loading mechanism, adapted to enable the release of said disinfecting agents through said nozzle.

It is another object of the present invention to provide the system as defined above, additionally comprising a spray cone (503) in liquid communication with said nozzle, adapted to direct said disinfecting agents spray from said spray pump onto said stethoscope head.

It is another object of the present invention to provide the system as defined above, wherein said disinfecting agents are selected from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

It is another object of the present invention to provide the system as defined above, additionally comprising a clip (208) coupled to said back face, adapted to attach the device to a physician pocket.

It is another object of the present invention to provide the system as defined above, additionally comprising a clip coupled to said back face, adapted to attach the device to a stethoscope head.

It is another object of the present invention to provide a system for disinfecting a stethoscope head; wherein said system comprising:
a. a front cover (1002) having an aperture (1013) for engagement with said stethoscope head, and a back cover (1005);
b. a roll of disinfectant-impregnated material (1004) rolled around a supply spindle (1009) and an uptake spindle (1008);
c. a sprocket (1010) in physical communication with said supply spindle (1009), adapted to advance said roll of disinfectant-impregnated material (1004) when said sprocket (1010) is rotated;
d. a gear wheel (1011) coupled to said sprocket (1010);
e. a panel (1001) in physical communication with said front cover (1002); said panel having means (1014) adapted to rotate said gear wheel (1011) such that said sprocket (1010) is rotated when said front cover (1002) is opened.

It is another object of the present invention to provide the system as defined above, wherein said means (1014) is selected from a group consisting of a hinge, arm, spring, band, belt, gear wheel, linear gear.

It is another object of the present invention to provide the system as defined above, additionally comprising side panels (1003 and 1007) adapted to provide structural support.

It is another object of the present invention to provide the system as defined above, wherein said side panels (1003) are provided with a backing (1012) adapted to maintain said roll of disinfectant-impregnated material (1004) in place when the stethoscope head is inserted into said aperture (1013).

It is another object of the present invention to provide the system as defined above, additionally comprising a clip (1006) adapted to allow easy transportation of said system by attaching said system to a physician pocket.

It is another object of the present invention to provide the system as defined above, additionally comprising a clip coupled to said back face, adapted to attach the device to a stethoscope head.

It is another object of the present invention to provide a system adapted to disinfect a stethoscope head (1209); wherein said system comprises: (a) a capsule (1203) having a front side and a back side, containing a disinfectant solution and provided with multiplicity of perforations (1210) on said front side, and (b) a portable dispenser adapted for dispensing said disinfectant solution, said dispenser comprising:
a. a front face (1202) having an aperture adapted to accommodate said stethoscope head (1209), and a back face (1201);
b. a backing plate (1204) adapted to support said capsule (1203);
c. a load spreading plate (1212) adapted to support said backing plate (1204);
d. a front cover (1211) adapted to be pressed against said capsule (1203) and provided with bristles (1209) adapted to clean the surface of said stethoscope head (1209) when said head is pressed against said front cover (1211), and provided with perforations to allow said capsule to release said disinfectant onto said stethoscope head (1209;
e. support rods (1205) adapted to support said front cover (1211);
f. springs (1206) adapted to press against said support rods (1205);
g. a spring (1207) adapted to press against said load spreading plate (1212), where said spring (1207) provides more force than both springs (1206) in combination.

It is another object of the present invention to provide the system as defined above, additionally comprising a clip coupled to said back face (1201), adapted to attach the device to a shirt or pants pocket.

It is another object of the present invention to provide the system as defined above, additionally comprising a clip coupled to said back face (1201), adapted to attach the device to a stethoscope head.

It is another object of the present invention to provide the system as defined above, wherein said capsule is impregnated with a disinfecting agent taken from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

It is another object of the present invention to provide a method for disinfecting a stethoscope head (202). The method comprising steps selected inter alia from:
a. providing a plurality of adhesive sterilizing pads (204) housed within a spring loaded dispenser (302), said dispenser (302) comprising:
   i. a front face (203) having an aperture adapted to accommodate said stethoscope head (201), and a back face (207);
   ii. a cover (202) for encasing said aperture, said cover is at least partially removable from said front face during use of said system;
   iii. a container (205) adapted to accommodate said disposable pads (204);
b. inserting said stethoscope head into said aperture within said dispenser;
c. adhering the frontmost of said adhesive sterilizing pads (204) to said stethoscope head; thereby disinfecting said stethoscope head.

It is another object of the present invention to provide the method as defined above, additionally comprising step of removing said stethoscope head from said dispenser.

It is another object of the present invention to provide the method as defined above, additionally comprising step of removing said adhesive sterilizing pad from said stethoscope head.

It is another object of the present invention to provide the method as defined above, additionally comprising step of impregnating said disposable pads with a disinfecting agent taken from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

It is another object of the present invention to provide the method as defined above, additionally comprising step of impregnating said pads are with an adhesive agent selected from a group including elastomers, polymers, thermoplastics, thermosetting adhesives, cement, mucilage, glue, paste, cyanoacrylates, methacrylates, urethanes, epoxies, serum albumen, agar, algin, collagen, casein, starch, resins, and bioadhesives, and combinations thereof.

It is another object of the present invention to provide a method for disinfecting a stethoscope head with disinfecting agents. The method comprising steps selected inter alia from:
a. providing a mechanism having:
   i. an entrance portion (509) adapted to accommodate said stethoscope head;
   ii. an exit portion (510) adapted to extract said stethoscope head; said entrance portion and exit portion are in physical communication so as to enable said stethoscope head to be conducted from said entrance portion to said exit portion;
   iii. a reservoir (504) accommodating said disinfecting agents;
   iv. a spraying mechanism adapted to spray said stethoscope head with said disinfecting agent when said stethoscope head is conducted from said entrance portion to said exit portion
b. inserting said stethoscope head into said entrance portion (509);
c. conducting said stethoscope head to said exit portion (510), thereby activating said spraying mechanism and disinfecting said stethoscope head.
d. removing said stethoscope head from said exit port, triggering said trigger level and thus causing said spring loaded mechanism to discharge said disinfecting agent onto said stethoscope head

It is another object of the present invention to provide the method as defined above, additionally comprising the step of removing said stethoscope head from said exit portion (510).

It is another object of the present invention to provide the method as defined above, additionally comprising the step of providing a spray cone (503) for directing said disinfecting agents spray onto said stethoscope head.

It is another object of the present invention to provide the method as defined above, additionally comprising the step of selecting said disinfecting agent from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

It is another object of the present invention to provide a method for disinfecting a stethoscope head. The methods comprising steps selected inter alia from:
a. providing a mechanism having:
   i. a front cover (1002) having an aperture (1013) for engagement with said stethoscope head; and, a back cover (1005);
   ii. a roll of disinfectant-impregnated material (1004) rolled around a supply spindle (1009) and an uptake spindle (1008);
   iii. a sprocket (1010) in physical communication with said supply spindle (1009), adapted to advance said roll of disinfectant-impregnated material (1004) when said sprocket (1010) is rotated;
   iv. a gear wheel (1011) coupled to said sprocket (1010);
   v. a panel (1001) in physical communication with said front cover (1002); said panel having means (1014) adapted to rotate said gear wheel (1011) such that said sprocket (1010) is rolled;
b. inserting said stethoscope head into said aperture (1013);
c. physical contacting said stethoscope head with said roll of disinfectant-impregnated material (1004); thereby disinfecting said stethoscope head;
d. closing said door (1001); whereby portable disinfection of a stethoscope head with a clean surface of disinfectant-impregnated material is provided.

It is another object of the present invention to provide the method as defined above, additionally comprising a step of activating said means (1014) by opening said panel (1001) thereby rotating said gear wheel (1011), said sprocket (1010) and said supply spindle (1009).

It is another object of the present invention to provide the method as defined above, additionally comprising a step of rolling said roll of disinfectant-impregnated material (1004).

It is another object of the present invention to provide the method as defined above, additionally comprising a step of removing said stethoscope head from said aperture (1013).

It is another object of the present invention to provide the method as defined above, additionally comprising a step of closing said panel (1001).

It is another object of the present invention to provide the method as defined above, additionally comprising a step of impregnating said disposable pads with a disinfecting agent selected from a group consisting of solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

It is another object of the present invention to provide a method for disinfecting a stethoscope head (1209). The method comprising steps selected inter alia from:
a. providing a mechanism having a capsule (1203) containing a disinfectant solution and provided with perforations (1210) on its front side, and a portable dispenser adapted for dispensing said disinfectant solution, said dispenser comprising:
   i. a front face (1202) having an aperture adapted to accommodate said stethoscope head (1209), and a back face (1201);
   ii. a backing plate (1204) adapted to support said capsule (1203);
   iii. a load spreading plate (1212) adapted to support said backing plate (1204);
   iv. a front cover (1211) adapted to be pressed against said capsule (1203) and provided with bristles (1209) adapted to clean the surface of said stethoscope head (1209) when said stethoscope head (1208) head is pressed against said front cover (1211), and provided with perforations to allow said capsule to release said disinfectant onto said stethoscope head (1209;
   v. support rods (1205) adapted to support said front cover (1211);
   vi. spings (1206) adapted to press against said support rods (1205);
   vii. a spring (1207) adapted to press against said load spreading plate (1212), where said spring (1207) provides more force than both springs (1206) in combination; and,
b. pressing said stethoscope head against said front cover (1211), pressurizing said capsule (1203) and thus pushing said disinfectant solution out of said perforations (1210), while simultaneously wiping said stethoscope head (1208) against said bristles (1209).

It is another object of the present invention to provide the method as defined above, additionally comprising a step of providing a clip coupled to said back face (1201), adapted to attach the device to a shirt or pants pocket.

It is still an object of the present invention to provide the method as defined above, additionally comprising a step of providing a clip coupled to said back face (1201), adapted to attach the device to a stethoscope head.

It is lastly an object of the present invention to provide the method as defined above, additionally comprising a step of providing said capsule with a disinfecting agent taken from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a stethoscope disinfection device.

In the following detailed description, numerous specific details are set forth in order to provide a thorough understanding of embodiments of the present invention. However, those skilled in the art will understand that such embodiments may be practiced without these specific details. Reference throughout this specification to “one embodiment” or “an embodiment” means that a particular feature, structure, or characteristic described in connection with the embodiment is included in at least one embodiment of the invention. Thus, the appearances of the phrases “in one embodiment” or “in an embodiment” in various places throughout this specification are not necessarily all referring to the same embodiment or invention. Furthermore, the particular features, structures, or characteristics may be combined in any suitable manner in one or more embodiments. Lastly, the terms “comprising”, “including”, “having”, and the like, as used in the present application, are intended to be synonymous.

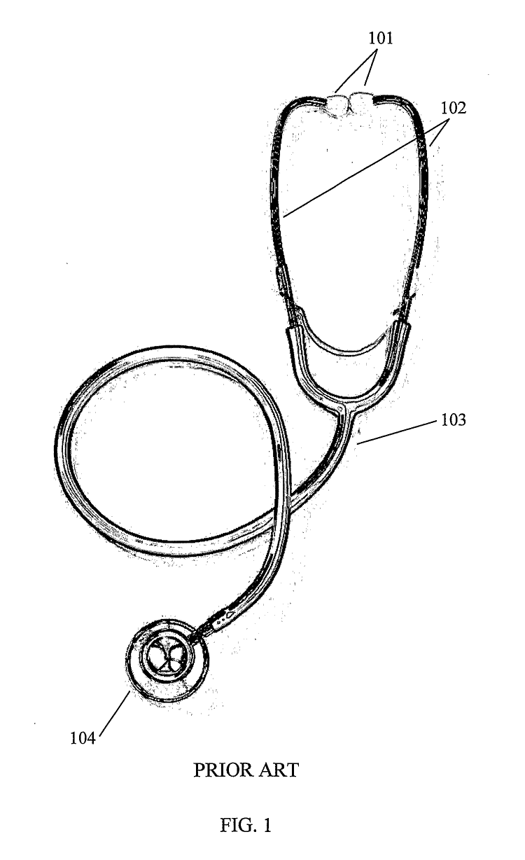

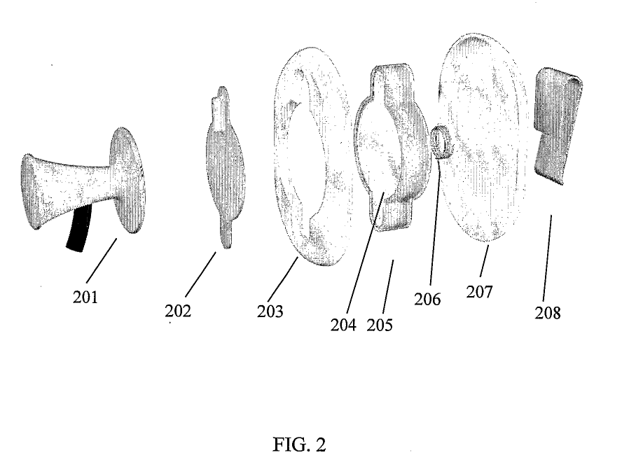

The term ‘stethoscope head’ refers to the end-most section of a stethoscope that contacts a patient's body (see 104 of FIG. 1).

The term ‘adhesive disinfecting pad’ refers to a pad impregnated with both adhesive and disinfecting agents.

The term ‘plurality’ refers hereinafter to any integer number equal or higher 1, e.g., 2-10, especially 2-4.

The term ‘use’ of a stethoscope head refers to pressing the stethoscope head against a portion of a person's body, such as their chest, and listening o bodily sounds by means of said stethoscope head. When a stethoscope head is so used, it comes into physical contact with said person's body and is thus exposed to whatever germs, viruses, bacteria, etc. that might lie upon said person's skin.

The proposed invention relates to a compact, convenient, portable device for disinfection of stethoscopes. It is designed to be easily worn by a healthcare provider via a clip or other means. The head of the stethoscope fits into the device, and disinfection preferably takes place by a “post-it” type of disposable pad having adhesive and disinfection agents therein. We call this pad an adhesive disinfecting pad. The device also serves as a holder for the stethoscope head when the stethoscope is not in use. Chief among the various advantages of the device is that it allows a healthcare practitioner to perform fast and easy disinfection of the stethoscope before each patient who undergoes examination due to its portability. The healthcare practitioner simply inserts the stethoscope head into the device (which he may be wearing), and disinfection occurs instantly. The device is lightweight and convenient, and offers the doctor a way to perform stethoscope disinfection in a matter of seconds. Thus, disinfection becomes a habitual part the healthcare practitioner's routine, and patients will come to expect stethoscope disinfection as a matter of course. The adhesive disinfecting pads are held inside of the device, and each time the stethoscope is inserted into the device, a new pad sticks onto the stethoscope head and is subsequently discarded following removal of the stethoscope from the device. A disinfection mechanism such as this is not disclosed in any of the prior art. Moreover, this mechanism provides significant advantages over the prior art since, with each usage, both the doctor and the patient can be sure that proper disinfection has occurred. This mechanism has clear advantages over the sanitizing pads of U.S. Patent Application No. 2007/0080017, since, using that invention, the doctor has to massage the device in order to maximize the germ killing ability of the pad, and the use of multiple pads can become problematic, since, for example, the doctor may have to regularly check how much disinfecting agent remains in the pad(s) in order to know when the pad needs to be replaced or massaged. In contrast to this, using the proposed invention, an optimum amount of disinfecting agent is provided with each disposable adhesive pad, and the stack of pads is simply replaced when it runs out. Additionally, the overall design of the proposed invention, in which the doctor can wear the device directly on the shirt or jacket (instead of holding it in his pocket or affixing into the stethoscope tube, as in U.S. Patent Application No. 2007/0080017) provides an added degree of convenience and appeal.

With reference to FIG. 2 we elucidate the various parts of the mechanism of the invention. The stethoscope head 201 fits into the front receptacle 203 once the cover 202 has been rotated out of the way. This brings the stethoscope head 201 into contact with the frontmost adhesive disinfecting pad 204 in the pad cartridge 205. The back shell 207 of the device holds the adhesive disinfecting pads in place as well as providing a backing plate against which the spring 206 pushes, resulting in a force tending to push the adhesive pads onto the stethoscope head. A pocket clip 208 allows the unit to be worn hanging from a pocket, belt, or the like.

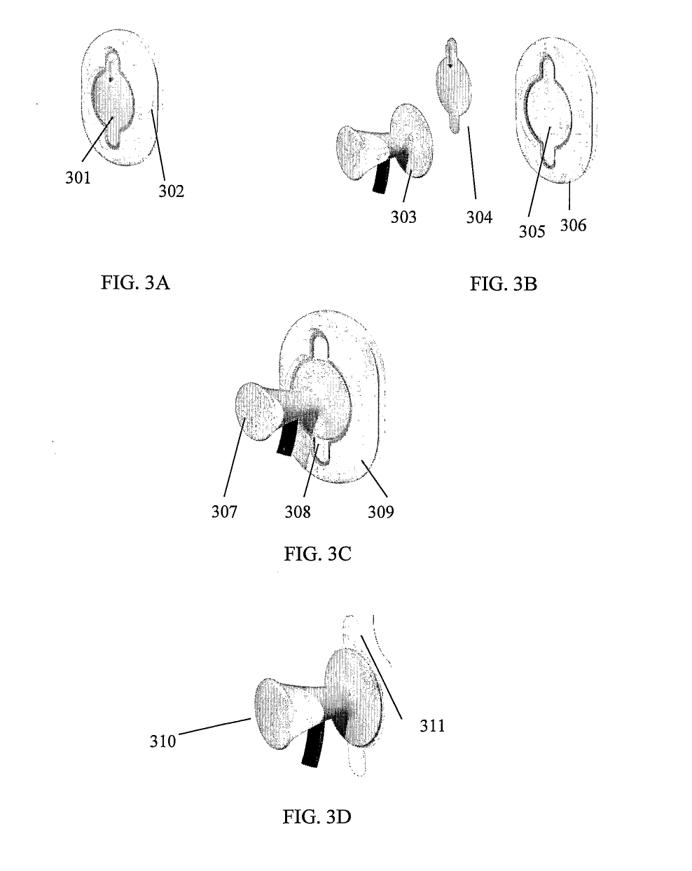

In FIG. 3 the sequence of events involved in using the invention are detailed. The front shell 302 at first holds the cover 301 in its closed position. When it is desired to disinfect a stethoscope, the cover 304 is rotated out of the way. This allows the stethoscope head 303 to be inserted into the dispenser body 306 and exposes the adhesive disinfecting pad 305. Once the stethoscope head 307 has been inserted into the dispenser body 309 it is forced into contact with a disinfecting pad 308. Due to the adhesive nature of this pad, when the stethoscope head 310 is removed from the dispenser, the adhesive pad 311 adheres to the stethoscope head. It can be subsequently peeled off and disposed of.

According to a preferred embodiment of the present invention, a dispenser is provided that is adapted to serially dispense pads have adhesive and disinfection agents absorbed within said pads. The amount of adhesive and disinfecting agents in said pads is controlled to provide a fixed dosage of adhesive and of disinfecting agents in each pad.

In one embodiment of the invention the dispenser is provided with a spring loaded mechanism for pushing the pads onto the stethoscope head.

In one embodiment of the invention the disinfecting pads are impregnated with a disinfecting agent taken from a group including solutions of citric acid, acetic acid, sulphamic acid, potassium monopersulfate, acidic iodophors, phenols, hypochlorites, iodophors such as iodine, quaternary ammonium, formaldehyde, alkali (lye), chlorhexidine, and oxidizing agents such as peroxide.

In one embodiment of the invention the pads are further impregnated with an adhesive agent selected from a group including elastomers, polymers, thermoplastics, thermosetting adhesives, cement, mucilage, glue, paste, cyanoacrylates, methacrylates, urethanes, epoxies, serum albumen, agar, algin, collagen, casein, starch, resins, bioadhesives, and combinations thereof.

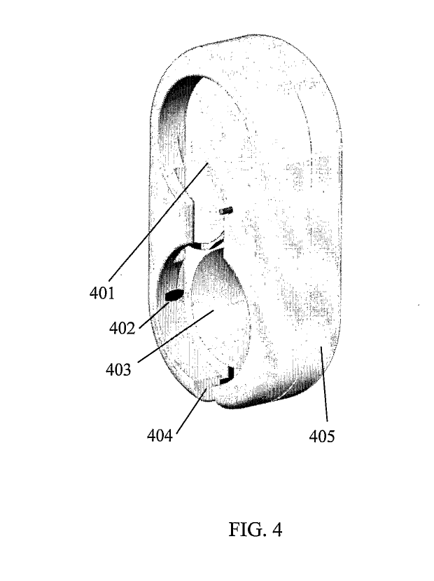

In another embodiment of the invention a spraying nozzle is employed that administers a fixed dose of disinfectant solution to the stethoscope head with each application. With reference to FIG. 4, a body 405 whose height is slightly larger than two stethoscope head diameters is provided with an entrance 401 and exit 404 for the stethoscope head. A cocking lever 402 is pushed down when the stethoscope is moved from entry to exit. This movement fills a pump with a fixed dose of disinfectant solution and compresses a pump spring. The dose is released on removing the stethoscope from the unit by means of releasing the trigger 404.

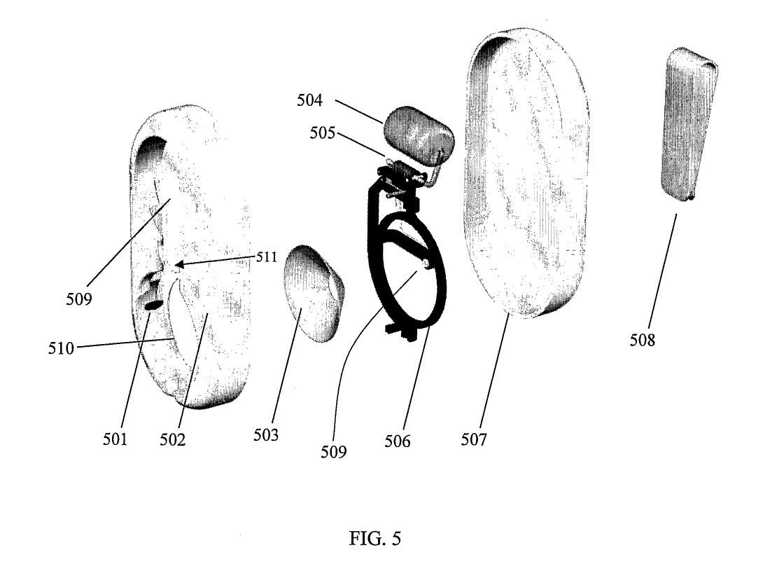

This mechanism is shown in greater detail in FIG. 5. This figure illustrates the trigger 501, the body 502, spray cone 503, disinfectant reservoir 504, disinfectant pump 505, trigger lever 506, back shell 507, and attachment clip 508. The stethoscope head is inserted into the entrance port 509, is conducted through the conduit 511, and removed from the exit port 510.

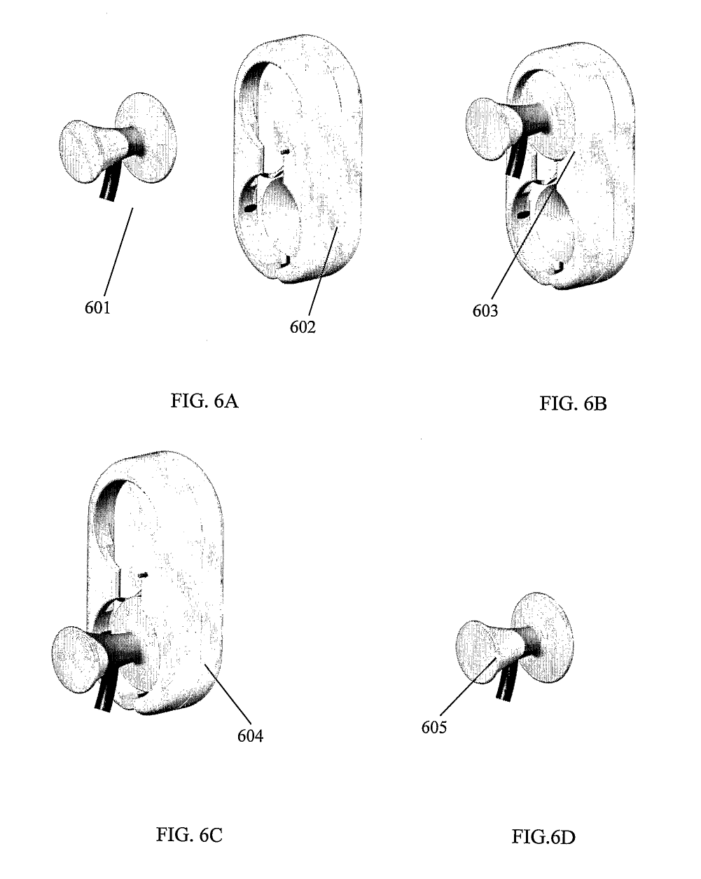

The method of use is illustrated in FIG. 6. The stethoscope 601 is pushed into the top hole 603 of the apparatus body 602. It is then forcibly slid down to the second opening 604 in the apparatus. By this action, as described above, a fixed amount of disinfectant solution is fed into a pump which is also spring loaded by this action. Upon removing the stethoscope head 605 a metered amount of solution is sprayed onto the stethoscope head due to the activation of the trigger mechanism 505 shown in FIG. 5.

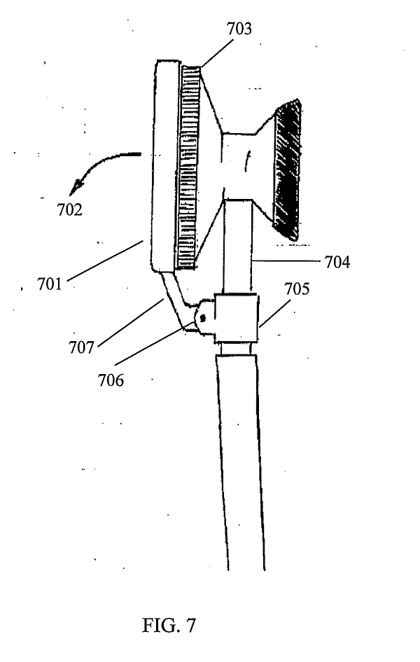

In another embodiment of the invention a swiveling cap is provided that protects and disinfects the stethoscope head when not in use. This embodiment is illustrated in FIG. 7. The cap 701 contains an absorbent pad impregnated with disinfectant material. This pad may be used multiple times before requiring replacement. The cap is held onto the stethoscope head tube 704 by means of an attachment ring 705. This ring is provided with a hinge 706 allowing the cap to swivel on arm 707 in the direction indicated 702, away from the stethoscope head 703. Thus when in use the cap does not interfere with stethoscope use. Upon finishing an examination, the healthcare practitioner swivels the cap 701 back onto the stethoscope head 703, thus disinfecting the stethoscope head and protecting it until the next use.

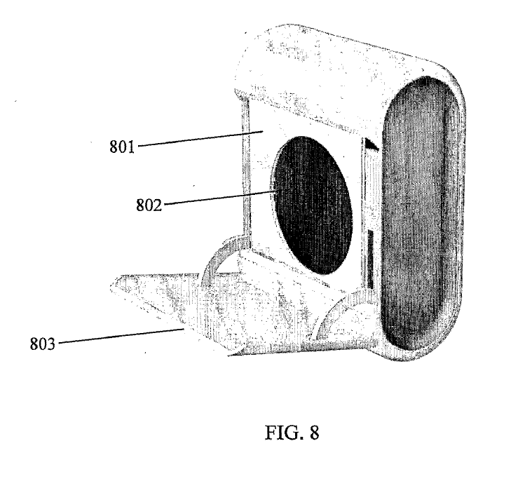

In another embodiment of the invention, a roll of disinfectant-impregnated material is contained within the housing of a unit adapted for this purpose. With reference to FIG. 8, the housing 801 contains the roll of disinfectant solution 802. Every time the hinged door 803 is opened, the roll 802 is advanced in a fashion similar to that used to advance the film in film cameras.

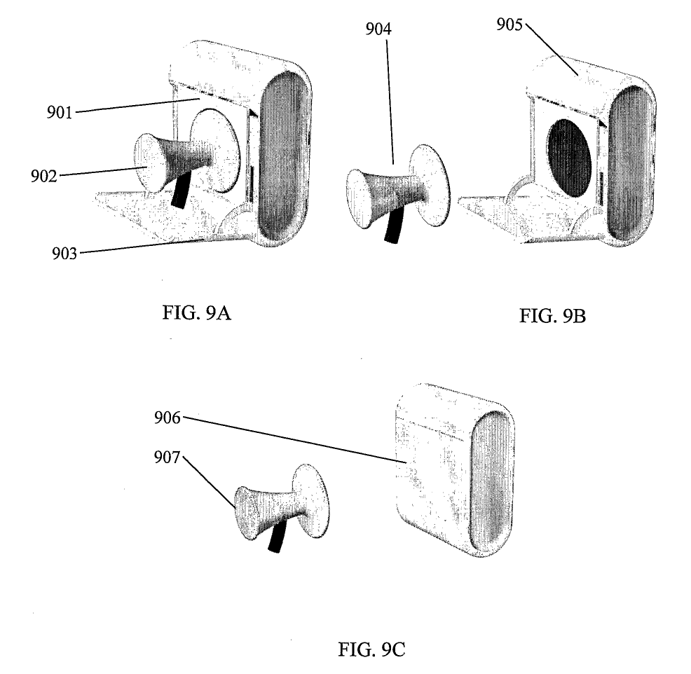

In FIG. 9 the method of use is presented. The housing 901 has been put into contact with a stethoscope head 902. The door of the housing 903 has been opened for this purpose. Once the head 904 is removed from the housing 905, the door 906 is closed. This has the dual purpose of advancing the roll of disinfectant film as well as keeping the disinfetant roll moist and clean.

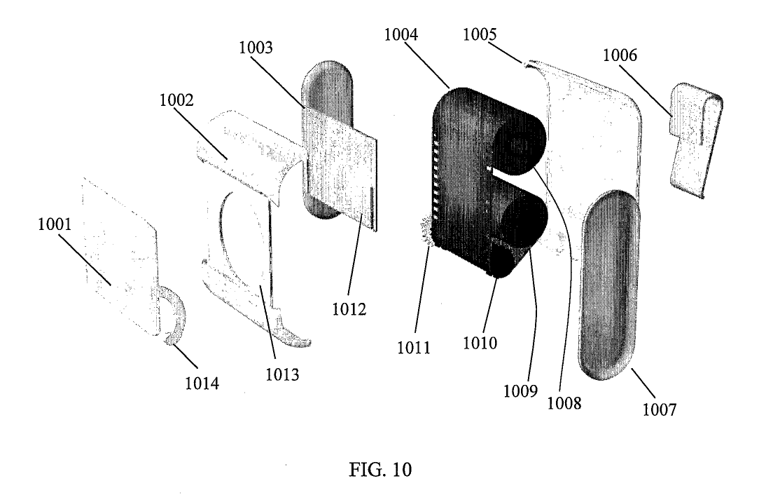

In FIG. 10 the detailed mechanism of this embodiment is presented. The door 1001 attaches to the front cover 1002 by means of a hinge 1014. The front cover has an opening 1013 adapted to allow insertion of a stethoscope head. The side panels 1003 and 1007 provide structural support. One of the side panels 1003 has a backing 1012 that keep the roll of disinfectant-impregnated material 1004 in place when the stethoscope head is inserted into the hole 1013. A back panel 1005 completes the main structure. A clip 1006 is optionally provided to allow easy transport. The roll 1004 is advanced by means of a hinged wheel 1011 that is in communication with a sprocket 1010. This hinged wheel 1011 is rotated by movement of the hinge 1014.

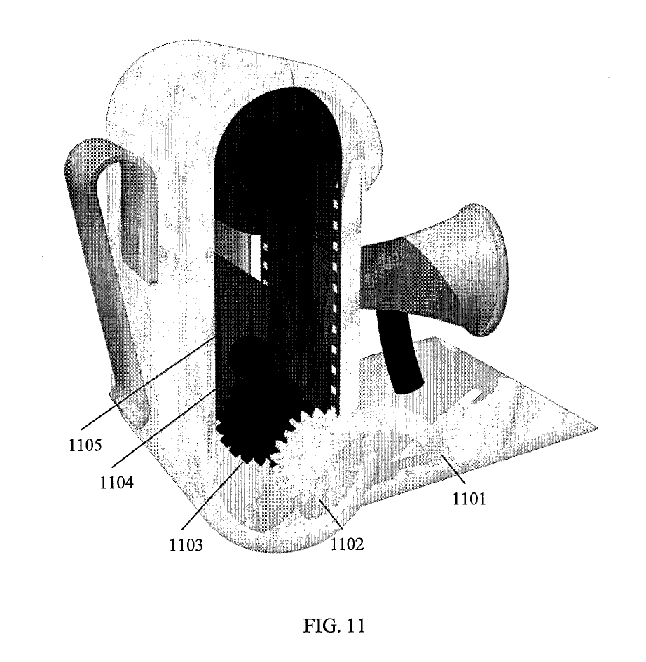

The hinge mechanism is shown more fully in FIG. 11. The hinge 1101 is attached to a gearwheel 1102. This gearwheel is incommunication with another gear 1103 which in turn is connected to the shaft 1104 of the roll 1105. By this means a gear reduction or increase may be effected, depending on the radii of the members 1102, 1103 and 1104.

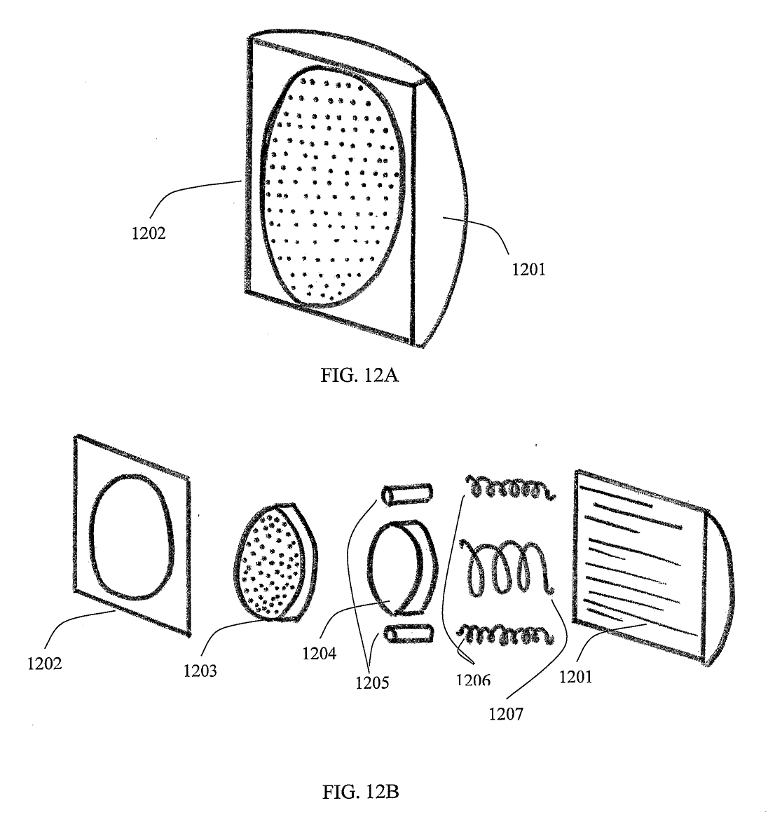

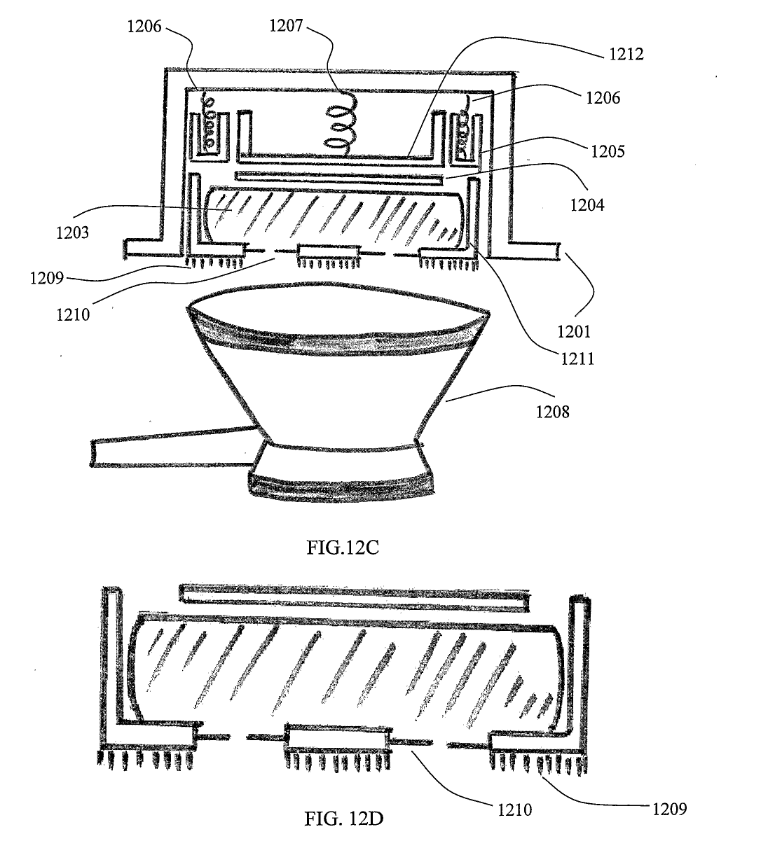

In another embodiment of the invention a capsule is employed to sterilize the stethoscope head. With reference to FIG. 12a, the overall structure of the device comprises a front cover 1202 and back cover 1201. In the exploded view of FIG. 12b further detail is evident. The front cover 1202 holds the capsule 1203 in place. This capsule is pressed against the front cover by a backing plate 1204. The support rods 1205 and backing plate 1204 are in contact with springs 1206 and 1207 respectively. In FIG. 12c the method of use can be discerned. The stethoscope head 1208 is pressed against the front cover of the device. The front face is provided with bristles 1209 that clean the stethoscope head by a wiping action, while the perforations 1210 allow fluid to be transmitted from the capsule 1202 to the stethoscope head. This will occur when the stethoscope head is pressed against the device. The springs 1206 are of a lower spring constant than the spring 1207. Therefore when the stethoscope head 1208 is forced against the device, the spring 1207 will resist this force, creating a corresponding pressure within the capsule 1202. This pressure will force the disinfectant solution within the capsule out the perforations 1210 and onto the stethoscope head 1208.

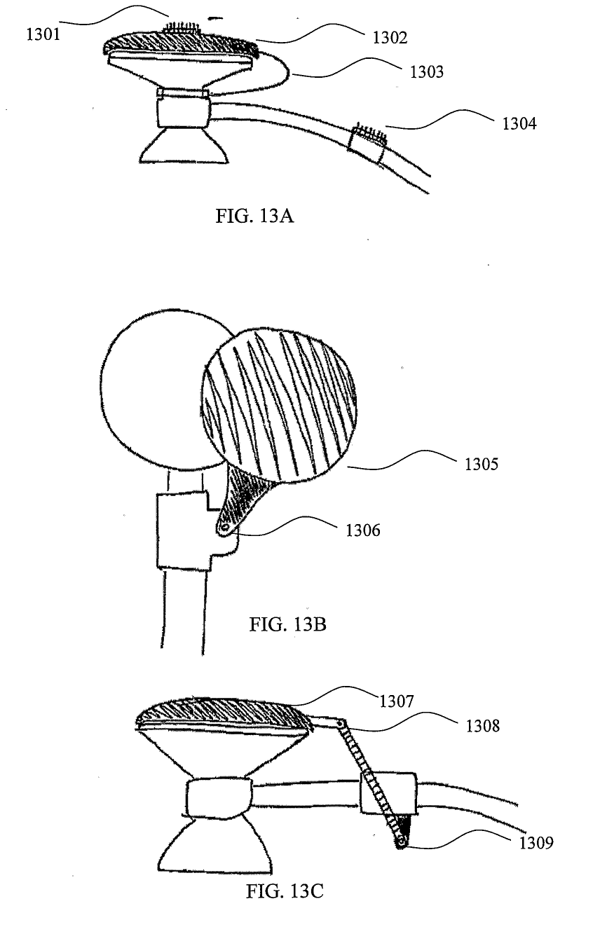

It should be pointed out that the other configurations are possible. In FIG. 13 several such embodiments are shown. Examples of such configurations are:
a. A velcro cover 1301 that can be attached to a velcro collar 1304, and retained by a retaining cord 1303.
b. A cover 1305 that is anchored to the stethoscope head and in a direction parallel to it by means of a hinge 1306.
c. A cover 1307 that is anchored to the stethoscope head by means of a hinged arm 1308 attached to a hinged collar 1309.

Additionally it should be pointed out that the disinfectant-containing pad placed on the inner part of the cover may be used many times before it should be replaced by a new one.

We note that each of the described above embodiments could be either portable (i.e. coupled to the physician pocked) or stationary.

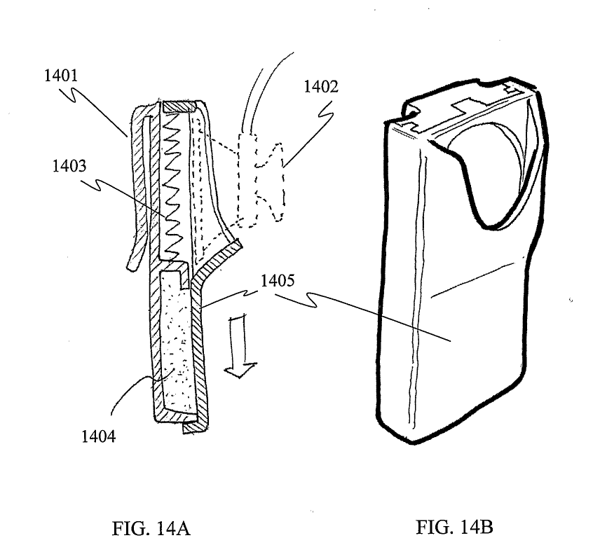

In another embodiment of the invention, detergent which is absorbed in a sponge comprises the disinfection mechanism. The sponge is held in a sealed container that contains a special lid mechanism, as shown in FIG. 14a, b. This embodiment is shown with a clip 1401 for attaching the device to a pocket or the like, a spring member 1403 to provide a reaction force against the stethoscope head 1402 when inserted, and disinfectant material in the sponge 1404. In FIG. 14a the device is seen from the side while in FIG. 14b the device is seen in perspective. While the device is not activated the special lid 1405 seals the sponge, preventing the disinfection detergent evaporation.

Activation of the device takes place using a single hand movement, in the direction of the arrow in FIG. 14a. The stethoscope's head is pulled down, removing the special lid 1405, revealing the disinfecting sponge directly on the stethoscope's head.

While removing the stethoscope, an incorporated spring 1403 pushes the lid back to its closed position.

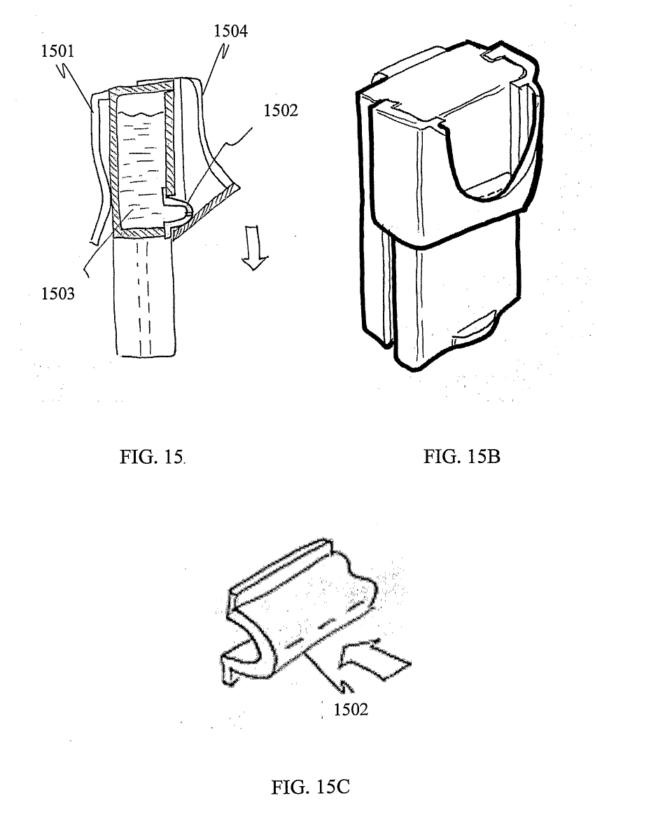

In another embodiment illustrated in FIG. 15a-c, a silicone nozzle 1502 and a reservoir of liquid detergent 1503 comprises the disinfection mechanism. The disinfection liquid 1503 is held in a sealed container that contains a special lid mechanism. The special lid is made out of a “baby bottle” like silicone nozzle 1502. When the stethoscope's head is pushed against the nozzle, the nozzle's micro-pores open, allowing the disinfecting liquid to spread over the stethoscope's head. In FIG. 15a the device is presented in side-view, while in FIG. 15b an isometric view is shown. In FIG. 15c the silicone nozzle 1502 is shown in close-up isometric view.

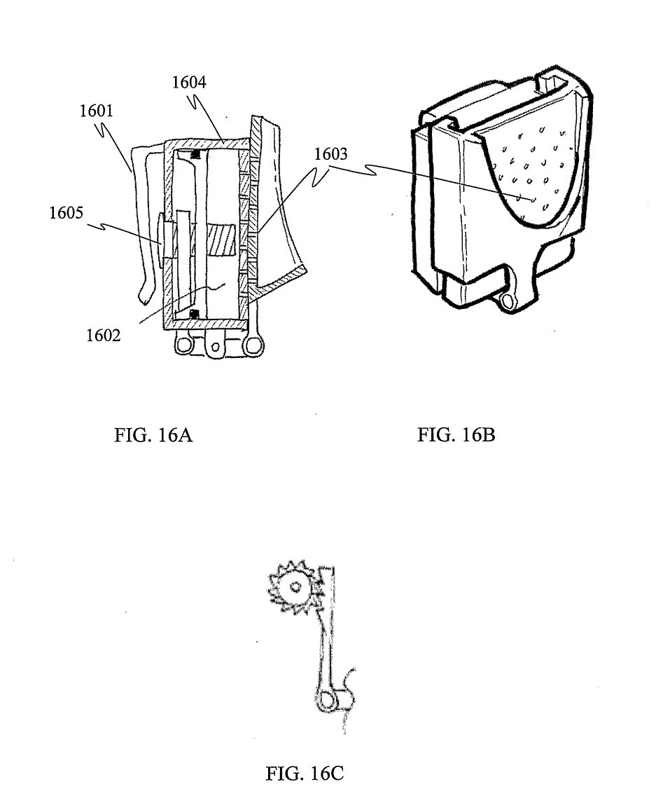

In another embodiment of the invention shown in FIG. 16a-c, a gel detergent and two grid like plates 1602, 1603 comprise the disinfection mechanism. The disinfecting gel is held in a sealed container 1604 that contains a special lid mechanism. The special lid is made out of two grid like plates 1602, 1603, that are positioned one in front of the other. When the stethoscope's head pulls the outer grid 1603 down, the openings of both grid's align, allowing the disinfecting gel to spread over the stethoscope's head. A special screw mechanism 1605 pushes the same amount of gel every use. A ratchet-and-pawl such as shown in FIG. 16c may be employed for this purpose.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to and with various modifications as are suited to the particular use contemplated. All such modifications and variations are within the scope of the invention as determined by the appended claims when interpreted in accordance with the breadth they are fairly, legally, and equitably entitled.

1-41. (canceled)

42. A system adapted to disinfect a stethoscope head (202) comprising: (a) a plurality of disposable pads (204) having adhesive and disinfection agents absorbed therein, said pads being adapted to adhere to and disinfect said stethoscope head; and (b) a portable dispenser adapted for serially dispensing said disposable pads, said dispenser comprising:
a. a front face (203) having an aperture adapted to accommodate said stethoscope head (201), and a back face (207);
b. a cover (202) for encasing said aperture, said cover being at least partially removable from said front face during use of said system;
c. a container (205) adapted to accommodate said disposable pads (204).

43. The system according to claim 42, additionally comprising a spring (206) coupled to said back face and to said container, adapted to push said disposable pads in the direction of said stethoscope head.

44. The system according to claim 42, additionally comprising a clip (208) coupled to said back face, adapted to attach the device to at least one selected from a group consisting of a physician pocket, a stethoscope head and any combination thereof.

45. The system according to claim 42, wherein said disposable pads are impregnated with a disinfecting agent selected from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

46. The system according to claim 42, wherein said pads are impregnated with an adhesive agent selected from a group including elastomers, polymers, thermoplastics, thermosetting adhesives, cement, mucilage, glue, paste, cyanoacrylates, methacrylates, urethanes, epoxies, serum albumen, agar, algin, collagen, casein, starch, resins, and bioadhesives, and combinations thereof.

47. A system adapted to disinfect a stethoscope head with disinfecting agents; wherein said system comprising:
a. an entrance portion (509) adapted to accommodate said stethoscope head;
b. an exit portion (510) adapted to extract said stethoscope head; said entrance portion and exit portion are in physical communication so as to enable said stethoscope head to be conducted from said entrance portion to said exit portion;
c. a reservoir (504) accommodating said disinfecting agents;
d. a spraying mechanism adapted to spray said stethoscope head with said disinfecting agent when said stethoscope head is conducted from said entrance portion to said exit portion.

48. The system according to claim 47, wherein said spraying mechanism additionally comprising:
a. a spray pump (505) having an inlet pipe coupled to said reservoir (504); and, an outlet pipe having at least one nozzle (509);
b. a spring loading mechanism in communication with said spray pump adapted to compress said spray pump when said stethoscope head is conducted from said entrance portion to said exit portion;
c. a trigger lever (506) in liquid communication with said spring loading mechanism, adapted to enable the release of said disinfecting agents through said nozzle.

49. The system according to claim 47, additionally comprising a spray cone (503) in liquid communication with said nozzle, adapted to direct said disinfecting agents spray from said spray pump onto said stethoscope head.

50. The system according to claim 47, wherein said disinfecting agents are selected from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

51. The system according to claim 47, additionally comprising a clip (208) coupled to said back face, adapted to attach the device to at least one selected from a group consisting of a physician pocket, a stethoscope head, and any combination thereof.

52. A system for disinfecting a stethoscope head; wherein said system comprising:
a. a front cover (1002) having an aperture (1013) for engagement with said stethoscope head, and a back cover (1005);
b. a roll of disinfectant-impregnated material (1004) rolled around a supply spindle (1009) and an uptake spindle (1008);
c. a sprocket (1010) in physical communication with said supply spindle (1009), adapted to advance said roll of disinfectant-impregnated material (1004) when said sprocket (1010) is rotated;
d. a gear wheel (1011) coupled to said sprocket (1010);
e. a panel (1001) in physical communication with said front cover (1002); said panel having means (1014) adapted to rotate said gear wheel (1011) such that said sprocket (1010) is rotated when said front cover (1002) is opened.

53. The system according to claim 52, wherein said means (1014) is selected from a group consisting of a hinge, arm, spring, band, belt, gear wheel, linear gear.

54. The system according to claim 52, additionally comprising side panels (1003 and 1007) adapted to provide structural support.

55. The system according to claim 52, wherein said side panels (1003) are provided with a backing (1012) adapted to maintain said roll of disinfectant-impregnated material (1004) in place when the stethoscope head is inserted into said aperture (1013).

56. The system according to claim 52, additionally comprising a clip (1006) adapted to allow easy transportation of said system by attaching said system to a physician pocket.

57. The system according to claim 52, additionally comprising a clip coupled to said back face, adapted to attach the device to a stethoscope head.

58. A system adapted to disinfect a stethoscope head (1209); wherein said system comprises: (a) a capsule (1203) having a front side and a back side, containing a disinfectant solution and provided with multiplicity of perforations (1210) on said front side, and (b) a portable dispenser adapted for dispensing said disinfectant solution, said dispenser comprising:
a. a front face (1202) having an aperture adapted to accommodate said stethoscope head (1209), and a back face (1201);
b. a backing plate (1204) adapted to support said capsule (1203);
c. a load spreading plate (1212) adapted to support said backing plate (1204);
d. a front cover (1211) adapted to be pressed against said capsule (1203) and provided with bristles (1209) adapted to clean the surface of said stethoscope head (1209) when said head is pressed against said front cover (1211), and provided with perforations to allow said capsule to release said disinfectant onto said stethoscope head (1209);
e. support rods (1205) adapted to support said front cover (1211);
f. springs (1206) adapted to press against said support rods (1205);
g. a spring (1207) adapted to press against said load spreading plate (1212), where said spring (1207) provides more force than both springs (1206) in combination.

59. The system according to claim 58, additionally comprising a clip coupled to said back face (1201), adapted to attach the device to at least one selected from a group consisting of shirt pocket, pants pocket, a stethoscope head and any combination thereof.

60. The system according to claim 58, wherein said capsule is impregnated with a disinfecting agent taken from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

61. A method for disinfecting a stethoscope head (202), said method comprising steps of:
a. providing a plurality of adhesive sterilizing pads (204) housed within a spring loaded dispenser (302), said dispenser (302) comprising:
i. a front face (203) having an aperture adapted to accommodate said stethoscope head (201), and a back face (207);
ii. a cover (202) for encasing said aperture, said cover is at least partially removable from said front face during use of said system;
iii. a container (205) adapted to accommodate said disposable pads (204);
b. inserting said stethoscope head into said aperture within said dispenser;
c. adhering the frontmost of said adhesive sterilizing pads (204) to said stethoscope head; thereby disinfecting said stethoscope head.

62. The method of claim 61, additionally comprising step of removing said stethoscope head from said dispenser.

63. The method of claim 61, additionally comprising step of removing said adhesive sterilizing pad from said stethoscope head.

64. The method of claim 61, additionally comprising step of impregnating said disposable pads with a disinfecting agent taken from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

65. The method of claim 61, additionally comprising step of impregnating said pads are with an adhesive agent selected from a group including elastomers, polymers, thermoplastics, thermosetting adhesives, cement, mucilage, glue, paste, cyanoacrylates, methacrylates, urethanes, epoxies, serum albumen, agar, algin, collagen, casein, starch, resins, and bioadhesives, and combinations thereof.

66. A method for disinfecting a stethoscope head with disinfecting agents; said method comprising steps of:
a. providing a mechanism having:
i. an entrance portion (509) adapted to accommodate said stethoscope head;
ii. an exit portion (510) adapted to extract said stethoscope head; said entrance portion and exit portion are in physical communication so as to enable said stethoscope head to be conducted from said entrance portion to said exit portion;
iii. a reservoir (504) accommodating said disinfecting agents;
iv. a spraying mechanism adapted to spray said stethoscope head with said disinfecting agent when said stethoscope head is conducted from said entrance portion to said exit portion
b. inserting said stethoscope head into said entrance portion (509);
c. conducting said stethoscope head to said exit portion (510), thereby activating said spraying mechanism and disinfecting said stethoscope head.
d. removing said stethoscope head from said exit port, triggering said trigger level and thus causing said spring loaded mechanism to discharge said disinfecting agent onto said stethoscope head

67. The method of claim 66, additionally comprising the step of removing said stethoscope head from said exit portion (510).

68. The method of claim 66, additionally comprising the step of providing a spray cone (503) for directing said disinfecting agents spray onto said stethoscope head.

69. The method of claim 66, additionally comprising the step of selecting said disinfecting agent from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

70. A method for disinfecting a stethoscope head; wherein said method comprising steps of:
a. providing a mechanism having:
i. a front cover (1002) having an aperture (1013) for engagement with said stethoscope head; and, a back cover (1005);
ii. a roll of disinfectant-impregnated material (1004) rolled around a supply spindle (1009) and an uptake spindle (1008);
iii. a sprocket (1010) in physical communication with said supply spindle (1009), adapted to advance said roll of disinfectant-impregnated material (1004) when said sprocket (1010) is rotated;
iv. a gear wheel (1011) coupled to said sprocket (1010);
v. a panel (1001) in physical communication with said front cover (1002); said panel having means (1014) adapted to rotate said gear wheel (1011) such that said sprocket (1010) is rolled;
b. inserting said stethoscope head into said aperture (1013);
c. physical contacting said stethoscope head with said roll of disinfectant-impregnated material (1004); thereby disinfecting said stethoscope head;
d. closing said door (1001);
whereby portable disinfection of a stethoscope head with a clean surface of disinfectant-impregnated material is provided.

71. The method according to claim 70, additionally comprising a step of activating said means (1014) by opening said panel (1001) thereby rotating said gear wheel (1011), said sprocket (1010) and said supply spindle (1009).

72. The method according to claim 70, additionally comprising a step of rolling said roll of disinfectant-impregnated material (1004).

73. The method according to claim 70, additionally comprising a step of removing said stethoscope head from said aperture (1013).

74. The method according to claim 70, additionally comprising a step of closing said panel (1001).

75. The method according to claim 70, additionally comprising a step of impregnating said disposable pads with a disinfecting agent selected from a group consisting of solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.

76. A method for disinfecting a stethoscope head (1209) wherein said method comprises steps of:
a. providing a mechanism having a capsule (1203) containing a disinfectant solution and provided with perforations (1210) on its front side, and a portable dispenser adapted for dispensing said disinfectant solution, said dispenser comprising:
i. a front face (1202) having an aperture adapted to accommodate said stethoscope head (1209), and a back face (1201);
ii. a backing plate (1204) adapted to support said capsule (1203);
iii. a load spreading plate (1212) adapted to support said backing plate (1204);
iv. a front cover (1211) adapted to be pressed against said capsule (1203) and provided with bristles (1209) adapted to clean the surface of said stethoscope head (1209) when said stethoscope head (1208) head is pressed against said front cover (1211), and provided with perforations to allow said capsule to release said disinfectant onto said stethoscope head (1209;
v. support rods (1205) adapted to support said front cover (1211);
vi. springs (1206) adapted to press against said support rods (1205);
vii. a spring (1207) adapted to press against said load spreading plate (1212), where said spring (1207) provides more force than both springs (1206) in combination; and,
b. pressing said stethoscope head against said front cover (1211), pressurizing said capsule (1203) and thus pushing said disinfectant solution out of said perforations (1210), while simultaneously wiping said stethoscope head (1208) against said bristles (1209).

77. The method according to claim 76, additionally comprising a step of providing a clip coupled to said back face (1201), adapted to attach the device to at least one selected from a group consisting of a shirt pocket, pants pocket, a stethoscope head and any combination thereof.

78. The system according to claim 76, additionally comprising a step of providing said capsule with a disinfecting agent taken from a group including solutions of citric acid, acetic acid, sulphamic acid, potassium monopersulfate, acidic iodophors, phenols, hypochlorites such as chlorine, iodophors such as iodine, quaternary ammonium, formaldehyde, alkali (lye), chlorhexidine, and oxidizing agents such as peroxide, and combinations thereof.
